(19) United States
(12) Patent Application Publication
(10) Pub. No.: US 2006/0196177 A1
(43) Pub. Date: Sep. 7, 2006

(54) Outboard engine system

(75) Inventors: Hiroki Tawa, Saitama (JP); Keiichirou Bungo, Saitama (JP); Makoto Yonezawa, Saitama (JP)

Correspondence Address: ARENT FOX PLLC, 1050 CONNECTICUT AVENUE, N.W., SUITE 400, WASHINGTON, DC, 20036, US

(73) Assignee: Honda Motor Co., Ltd.

(21) Appl. No.: 11/429,159

(22) Filed: May 8, 2006

Related U.S. Application Data
(63) Continuation of application No. 10/869,032, filed on Jun. 17, 2004.

(30) Foreign Application Priority Data
Jun. 25, 2003 (JP) 2003-180970

(51) Int. Cl. F01N 3/00; F01N 7/06; F01P 1/06; G01N 33/497

(52) U.S. Cl. 60/321; 60/276; 73/23.32; 123/41.31

(57) ABSTRACT

In an outboard engine system, an exhaust manifold fixed to a cylinder head of a vertical engine by a bolt includes single pipe portions and a collecting portion through which an exhaust gas flow. A water jacket, through which cooling water flows, is formed to surround a periphery of the collecting portion. An exhaust characteristics sensor for detecting an oxygen content of exhaust gas is mounted to an upper surface of the exhaust manifold. A water temperature sensor for detecting temperature of the cooling water is mounted to a side surface of the exhaust manifold.

### CROSS-REFERENCE TO RELATED APPLICATIONS

This is a Continuation Application, which claims the benefit of pending U.S. patent application Ser. No. 10/869,032, filed, Jun. 17, 2004. The disclosure of the prior application is hereby incorporated herein in its entirety by reference.

### BACKGROUND OF THE INVENTION

1. Field of the Invention

The present invention relates to an outboard engine system including a cylinder head forming a part of a combustion chamber and an exhaust passage component that is fixed to the cylinder head by a bolt. An exhaust passage in communication with the combustion chamber and a water jacket having cooling water flowing therethrough are formed inside the exhaust passage component.

2. Description of the Related Art

Japanese Patent Application Laid-Open Nos. 7-301135 and 10-47110 disclose outboard engine systems having an oxygen content sensor provided in an exhaust passage of an engine to detect the oxygen content in an exhaust gas. The oxygen content sensor helps control an air-fuel ratio of an air-fuel mixture to improve the output of the engine and suppress fuel consumption.

However, in the above-described conventional outboard engine systems, there is a relatively narrow space between the engine and the engine cover.

Therefore, in the case where a plurality of sensors are mounted to an exhaust passage component, such as an exhaust manifold of the engine, a sufficient mounting space cannot be defined without elaborate and complicated mounting of the sensors. Furthermore, the positioning of such sensors interferes with other devices and causes additional difficulties in establishing the layout of the engine components within and on the engine system.

### SUMMARY OF THE INVENTION

The present invention has been achieved in view of the aforementioned circumstances. It is an aspect of the present invention to define a space to simplify the manner in which a plurality of sensors are mounted on an exhaust passage component of an outboard engine system.

In order to achieve the above-described aspect, according to a first feature of the present invention, there is provided an outboard engine system including a cylinder head that forms a part of a combustion chamber and an exhaust passage component or manifold that is fixed to the cylinder head by a bolt. An exhaust passage in communication with a combustion chamber and a water jacket having cooling water passing therethrough are formed inside the exhaust passage component. Moreover, a plurality of sensors are mounted to an upper surface of the exhaust passage component and at least one side surface of the exhaust passage component, the side surface being different from a mounting surface to the cylinder head.

With the first feature, the sensors are mounted to the upper surface and the side surface of the exhaust passage component that is fixed to the cylinder head by the bolt. Therefore, the mounting space is more easily defined as compared or relative to the above-described conventional outboard engine systems wherein a plurality of sensors are mounted to the same surface. In addition, the side surface of the exhaust passage component to which the sensor is mounted differs from the side surface on which the exhaust passage component is fixed to the cylinder head by the bolt. Therefore, fixing the exhaust passage component to the cylinder head is not hindered by the sensor.

In addition to the first feature, according to a second feature of the present invention, the sensor mounted to the upper surface of the exhaust passage component is an exhaust characteristics sensor, which detects certain characteristics of an exhaust gas flowing through the exhaust passage. The exhaust characteristics sensor includes a pipe-shaped detection portion inserted downward into the exhaust passage and which has an opening in the side surface.

With the second feature, the exhaust characteristics sensor includes the opening in the side surface of the pipe-shaped detection portion, which is inserted downward in the exhaust passage. Therefore, even if an exhaust gas containing moisture enters the detection portion from the opening, the influence of the moisture on the detection accuracy of the sensor is minimized by preventing the moisture from staying in the detection portion.

In addition to the second feature, according to a third feature of the present invention, the exhaust passage component protrudes in a direction away from the cylinder block and the exhaust characteristics sensor is mounted to the upper surface at a position remote from the cylinder block.

With the third feature, a space for disposing various kinds of auxiliary devices is defined on the side surface of the cylinder block while maintaining a relatively smooth flow of the exhaust gas by securing the capacity of the exhaust passage component.

In addition to the first feature, according to a fourth feature of the present invention, the sensor mounted to the side surface of the exhaust passage component is a water temperature sensor which detects the temperature of the cooling water flowing through the water jacket.

According to the fourth feature, the water temperature sensor is mounted to the side surface of the exhaust passage component. Therefore, the temperature of the cooling water, flowing through the water jacket provided in the exhaust passage component, is detected.

In addition to the fourth feature, according to a fifth feature of the present invention, a cooling water outlet port is provided below the water jacket in the exhaust passage component. Also, the water temperature sensor is mounted below the cooling water outlet port.

With the fifth feature, the water temperature sensor on the side surface of the exhaust passage component is mounted at a position that is lower than the cooling water outlet port provided at the upper portion of the water jacket of the exhaust passage component. Therefore, the detection accuracy of the water temperature sensor is enhanced by ensuring that bubbles have difficulty staying in the upper portion of the water jacket.

The exhaust manifold in a below-described embodiment corresponds to the exhaust passage component of the present invention, wherein the collecting portion corresponds to the exhaust passage of the present invention, wherein the joints correspond to the cooling water outlet port of the present invention, wherein the exhaust characteristics sensor corresponds to the sensor of the present invention, wherein the water temperature sensor corresponds to the sensor of the present invention, and wherein the exhaust manifold cooling water jacket corresponds to the water jacket of the present invention.

The above-mentioned aspects, features, characteristics, and advantages of the present invention will become apparent from a preferred embodiment which will be described in detail below with reference to the attached drawings.

### BRIEF DESCRIPTION OF THE DRAWINGS

FIG. 1 is a partial cross-sectional side view of an outboard engine system according to an embodiment of the present invention;

FIG. 2 is an enlarged cross-sectional view of the system shown in FIG. 1 taken along line 2-2;

FIG. 3 is a view taken from the direction of arrow 3 in FIG. 2;

FIG. 4 is a view taken from the direction of arrow 4 in FIG. 3; and

FIG. 5 is a schematic diagram of an engine cooling system according to the present invention.

### DESCRIPTION OF THE PREFERRED EMBODIMENT

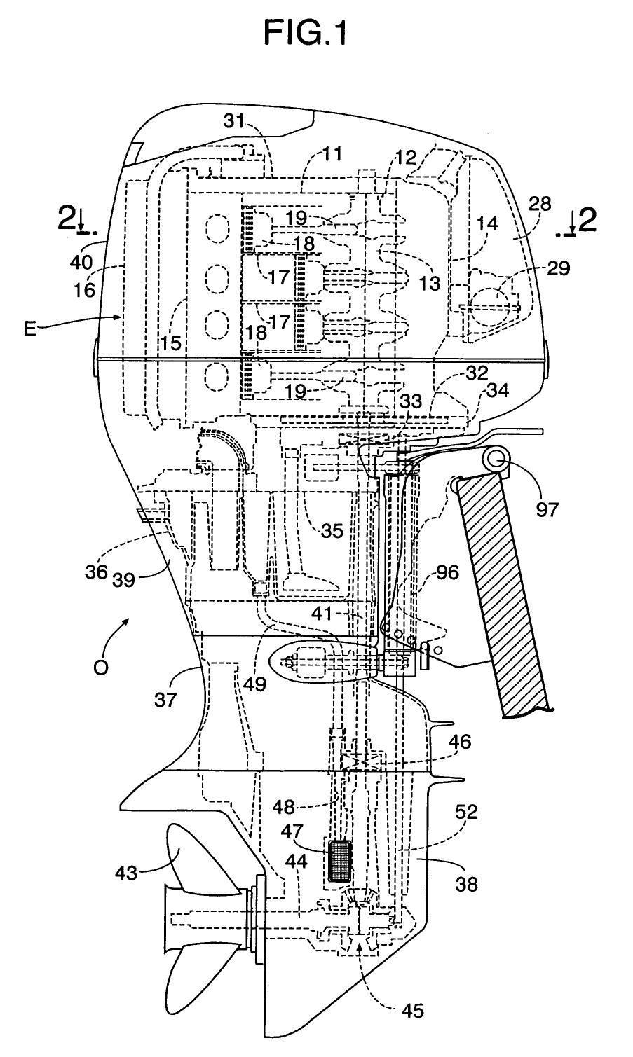

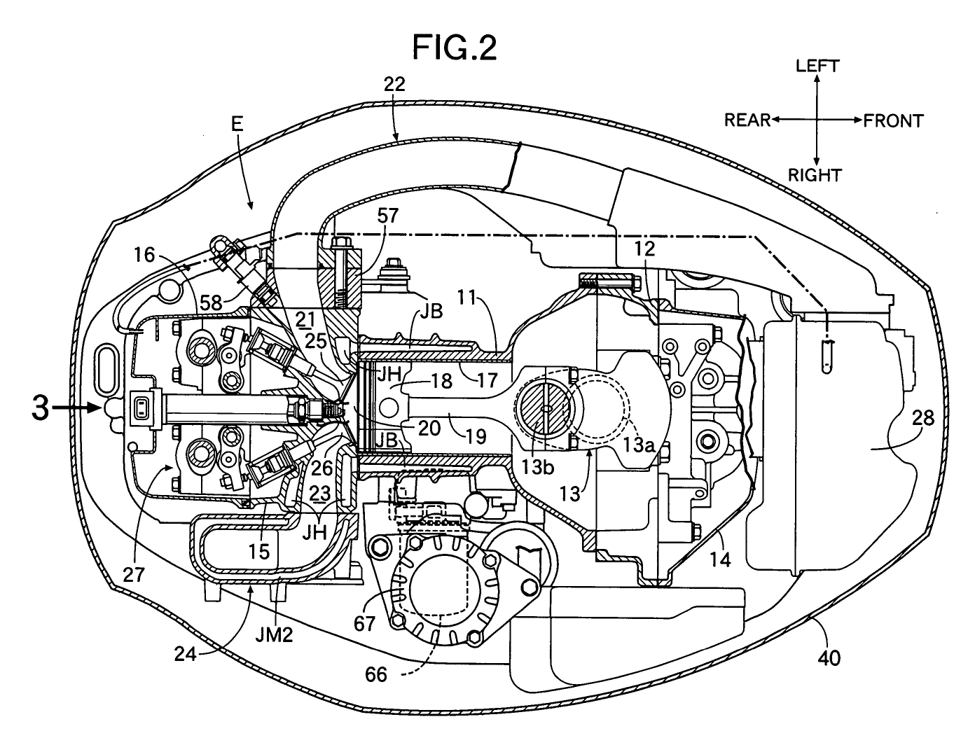

As shown in FIG. 1 and FIG. 2, an outboard engine system O is mounted to a ship body so as to make a steering movement in a lateral direction about a steering shaft 96, and a tilting movement in a vertical direction about a tilt shaft 97. An in-line four-cylinder four-stroke water-cooling vertical engine E mounted on an upper portion of the outboard engine system O includes a cylinder block 11; a lower block 12 connected to a front surface of the cylinder block 11; a crankshaft 13 disposed generally vertically and supported so that journals 13a are sandwiched between the cylinder block 11 and the lower block 12; a crankcase 14 connected to a front surface of the lower block 12; a cylinder head 15 connected to a rear surface of the cylinder block 11; and a head cover 16 connected to a rear surface of the cylinder head 15. Pistons 18 slidably fitted inside four sleeve-shaped cylinders 17 formed by enveloped-casting in the cylinder block 11 are respectively connected to crank pins 13b of the crankshaft 13 via connecting rods 19.

Combustion chambers 20, formed in the cylinder head 15 to be opposite the top surfaces of the pistons 18, are connected to an intake manifold 22 via intake ports 21 which are open to a left side surface of the cylinder head 15, namely, a port side in a traveling direction of the ship. The combustion chambers 20 are also connected to an exhaust passage 24 inside an engine room via exhaust ports 23 that open to a right side surface of the cylinder head 15. Intake valves 25 for opening and closing a downstream end of the intake ports 21 and exhaust valves 26 for opening and closing an upstream end of the exhaust ports 23 are driven to open and close by a DOHC-type valve moving mechanism 27 housed inside the head cover 16. An upstream side of the intake manifold 22 is disposed in front of the crankcase 14, connected to a throttle valve 29 fixed to a front surface, and is supplied with intake air through a silencer 28. Injectors 58 for injecting a fuel into the intake ports 21 are provided at an injector base 57 sandwiched between the cylinder head 15 and the intake manifold 22.

A chain cover 31 for housing a timing chain (not shown) that transmits a driving force of the crankshaft 13 to the valve moving mechanism 27 is connected to upper portions of the cylinder block 11, the lower block 12, the crankcase 14, and the cylinder head 15 of the engine E. An oil pump body 34 is connected to lower surfaces of the cylinder block 11, the lower block 12, and the crankcase 14. A mount case 35, an oil case 36, an extension case 37, and a gear case 38 are sequentially connected to a lower surface of the oil pump body 34.

The oil pump body 34 houses an oil pump 33 between a lower surface of the oil pump body 34 and an upper surface of the mount case 35. On the opposite side, a flywheel 32 is disposed between the oil pump body 34 and a lower surface of the cylinder block 11, and the like. A flywheel chamber and an oil pump chamber are defined by the oil pump body 34. The oil case 36, the mount case 35, and a periphery of a part of a lower side of the engine E are covered with an undercover 39 made of a synthetic resin. An upper part of the engine E is covered with an engine cover 40 made of a synthetic resin connected to an upper surface of the undercover 39.

A drive shaft 41 connected to a lower end of the crankshaft 13 penetrates through the pump body 34, the mount case 35 and the oil case 36, extends downward inside the extension case 37, and is connected to a front end of a propeller shaft 44. The shaft 44 includes a propeller 43 at a rear end and is supported at a gear case 38 in a longitudinal direction via a forward and reverse travel switching mechanism 45 operated by a shift rod 52. A lower water supply passage 48, which extends upward from a strainer 47 provided at the gear case 38, is connected to a cooling water pump 46 provided at the drive shaft 41. An upper water supply pipe 49, which extends upward from the water cooling pump 46, is connected to a cooling water supply passage 36b (see FIG. 7) provided in the oil case 36.

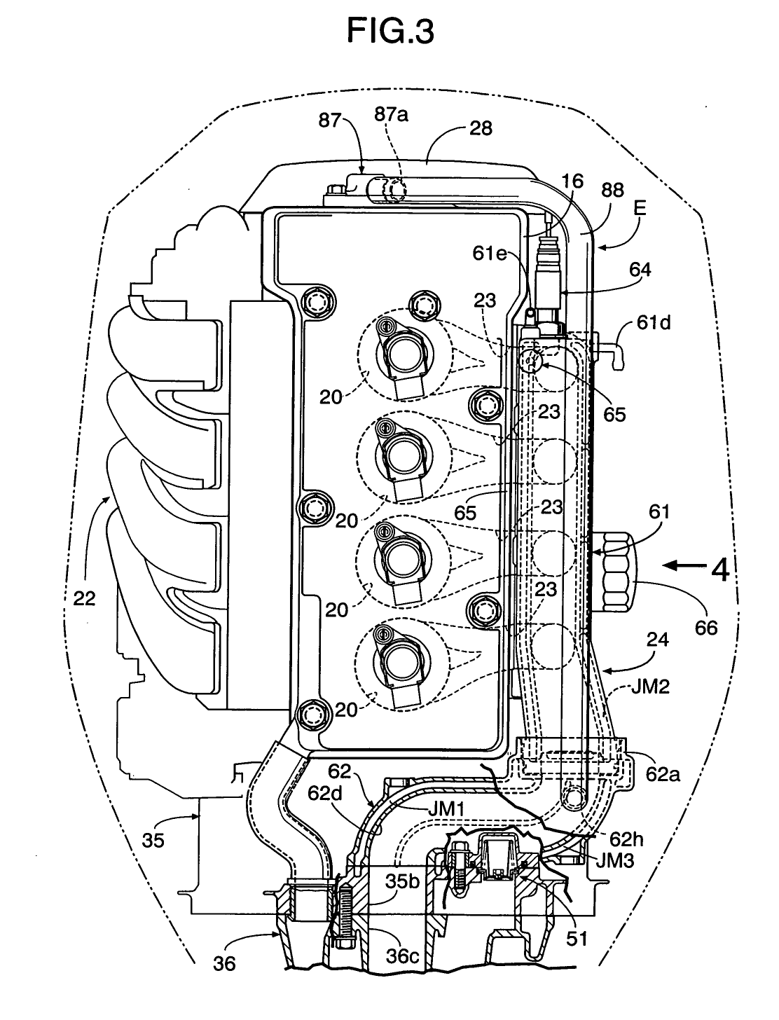

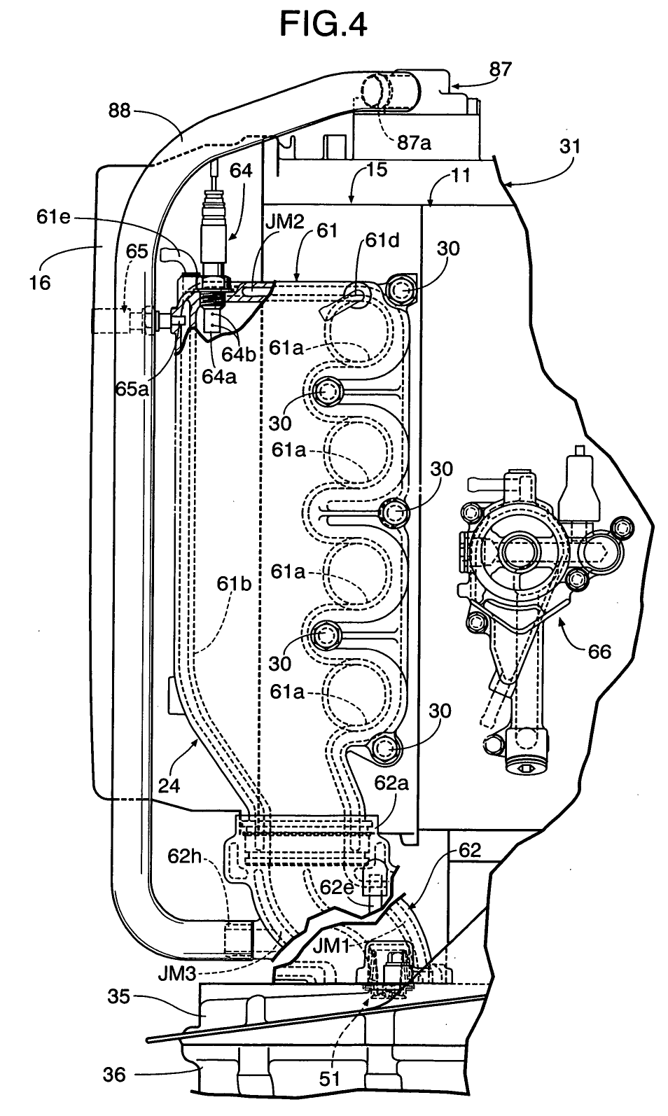

Next, a structure of an exhaust system and a cooling system of the engine E will be explained by reference to FIG. 2 to FIG. 5.

The exhaust passage means of the engine E is broadly divided into an exhaust passage 24 portion in an engine room and an exhaust chamber portion divided from the engine room. The exhaust passage 24 in the engine room includes: an exhaust manifold 61 having single pipe portions 61a, which are connected by bolts 30 to a right side surface of the cylinder head 15 and introduce exhaust gas from each of the combustion chambers 20, and a collecting part 61b in which the single pipe portions 61a are collected at the downstream regions of the single pipe portions 61a; and an exhaust guide 62 for guiding the exhaust gas to the outside of the engine room.

The exhaust guide 62 is connected to the upper surface of the mount case 35, which forms a partition wall of the engine room, and communicates with an exhaust passage 35b penetrating through the mount case 35. The exhaust passage 35b communicates with an exhaust chamber 63 (see FIG. 7) in the extension case 37 via an exhaust pipe portion 36c formed integrally with the oil case 36.

The exhaust manifold 61 includes four single pipe portions 61a, communicating with four exhaust ports 23, and the collecting part 61b where the single pipe portions 61a are integrally collected. The collecting part 61b extends in a direction away from the cylinder block 11 and is disposed along the cylinder head 15 and the head cover 16. The exhaust guide 62 is curved into an S-shape, and a lower end portion of the exhaust manifold 61 is fitted to an inner periphery of a connecting portion 62a having a large diameter at an upper end of the exhaust guide 62.

A first exhaust guide cooling water jacket JM1 for covering a half of the periphery of an upper surface side of the exhaust guide 62, and a second exhaust guide cooling water jacket JM3 for covering a half of the periphery of a lower surface side of the exhaust guide 62 are formed to surround the exhaust passage 62d. An exhaust manifold cooling water jacket JM2 is formed to surround a periphery of the exhaust manifold 61. When the lower end of the exhaust manifold 61 is fitted to an inner periphery of the connecting portion 62a of the exhaust guide 62, the exhaust manifold cooling water jacket JM2 of the exhaust manifold 61 and the first exhaust guide cooling water jacket JM1 of the exhaust guide 62 communicate with each other.

A connecting hole 62e of the first exhaust guide cooling water jacket JM1 communicates with an intermediate portion of a hose (not shown), which supplies cooling water to an oil filter 66 that will be described later, and supplies and discharges the cooling water when the engine stops.

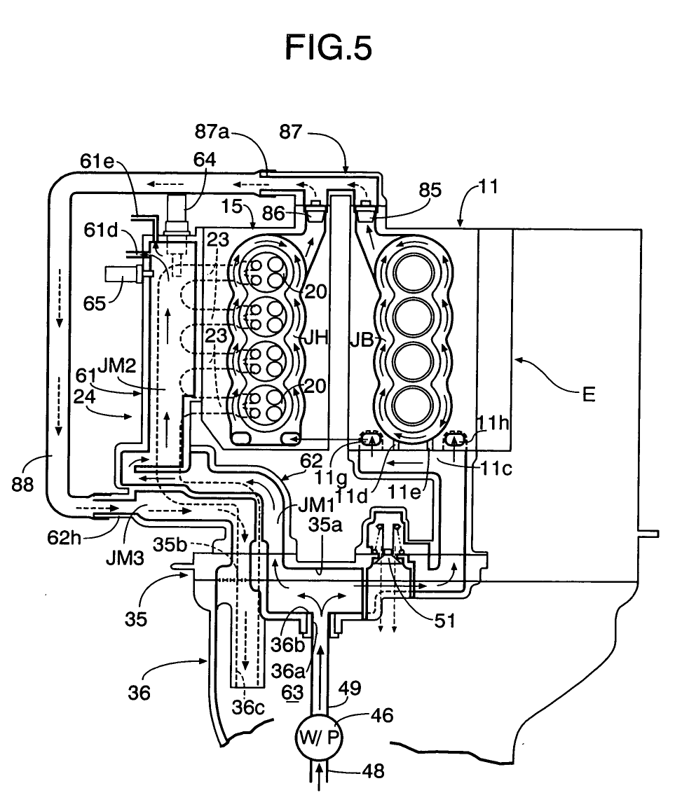

Next, referring primarily to FIG. 5, which shows a flow path of the cooling water, a structure and an operation regarding cooling of the entire engine E will be explained.

When the drive shaft 41 connected to the crankshaft 13 is rotated by operation of the engine E, the cooling pump 46 provided at the drive shaft 41 is operated to supply the cooling water, which is drawn up through the strainer 47, to the cooling water supply port 36a at a lower surface of the oil case 36 via the lower water supply passage 48 and the upper water supply pipe 49. The cooling water, which passes through the cooling water supply port 36a, flows into the cooling water supply passage 36b of the oil case 36 and the cooling water supply passage 35a of the mount case 35. A portion of the cooling water branching from the cooling water supply passage 36b and the cooling water supply passage 35a is supplied to the first exhaust guide cooling water jacket JM1 formed in the exhaust guide 62 of the exhaust passage 24 in the engine room and the exhaust manifold cooling water jacket JM2 formed in the exhaust manifold 61. The exhaust gas, which is discharged from the combustion chambers 20 of the cylinder head 15, is discharged to the exhaust chamber 63 via the single pipe portions 61a and the collecting portion 61b of the exhaust manifold 61, the exhaust passage 62d of the exhaust guide 62, the exhaust passage 35b of the mount case 35, and the exhaust pipe portion 36c of the oil case 36. In this process, the exhaust passage 24 in the engine room, which assumes a high temperature due to the exhaust gas, is cooled by the cooling water flowing through the first exhaust guide cooling water jacket JM1 and the exhaust manifold cooling water jacket JM2.

The cooling water, which flows upward through the first exhaust guide cooling water jacket JM1 and the exhaust manifold cooling water jacket JM2 to assume a raised temperature, is discharged from the joints 61d and 61e provided at the upper end of the exhaust manifold 61 through an unillustrated pipe or the like into the exhaust chamber 63.

Meanwhile, a portion of the cooling water having a low temperature, which is supplied to the cooling water supply passages 36b and 35a leading to the cooling water supply port 36a, flows into a lower end of a cylinder block cooling water jacket JB via two through-holes 11d and 11e opened to a cooling water supply passage 11c at the lower end of the cylinder block 11. A portion of the cooling water having a low temperature, which is supplied into the cooling water supply passages 36b and 35a, flows from the cooling water supply passage 11c at the lower end of the cylinder block 11 through two cooling water supply passages 11g and 11h into a lower end of a cylinder head cooling water jacket JH.

While warming-up the engine E, valves of a first thermostat 85, connected to the upper end of the cylinder block cooling water jacket JB, and a second thermostat 86, connected to the upper end of the cylinder head cooling water jacket JH, are closed. The cooling water in the cylinder block cooling water jacket JB and the cylinder head cooling water jacket JH stays without flowing, thus promoting warming-up of the engine E. In this case, the cooling water pump 46 continues to rotate, but the cooling water leaks from the periphery of the rubber impeller, wherein the cooling water pump 46 is substantially in an idling state.

When warming-up of the engine E is completed and the temperature of the cooling water rises, the valves of the first and the second thermostats 85 and 86 open, and the cooling water in the cylinder block cooling water jacket JB and the cooling water in the cylinder head cooling water jacket JH flow from a common joint 87a of a thermostat cover 87, through an exhaust pipe 88 and the joint 62h of the exhaust guide 62, and into the second exhaust guide cooling water jacket JM3. The cooling water, which cools the exhaust guide 62 while flowing through the second exhaust guide cooling water jacket JM3, passes downward through the mount case 35 and the oil case 36 before being discharged into the exhaust chamber 63.

An exhaust characteristics sensor 64 for measuring an oxygen content in an exhaust gas and controlling an air-fuel ratio is mounted at a position on an upper surface of the exhaust manifold 61, nearest to the head cover 16. The exhaust characteristics sensor 64 includes a pipe-shaped detecting portion 64a extending into the interior of the collecting portion 61b of the exhaust manifold 61. A plurality of openings 64b, through which the exhaust gas passes, are formed in a side surface of the detecting portion 64a. The exhaust characteristics sensor 64 is a linear type capable of successively detecting the oxygen content.

A water temperature sensor 65 for detecting the water temperature of cooling water is mounted to an upper portion of a side surface of the exhaust manifold 61 on the rear side. The water temperature sensor 65 includes a detection portion 65a extending into the interior of the exhaust manifold cooling water jacket JM2.

Since the exhaust characteristics sensor 64 is mounted on the upper surface of the exhaust manifold 61, and the water temperature sensor 65 is mounted to the side surface as described above, the mounting space is easily defined compared with the case where both the exhaust characteristics sensor 64 and the water temperature sensor 65 are mounted to the same surface of the exhaust manifold 61. In addition, the rear surface of the exhaust manifold 61, to which the water temperature sensor 65 is mounted, is different from the side surface on which the exhaust manifold 61 is fixed to the cylinder head 15 with the bolts 30. Therefore, mounting of the exhaust manifold 61 to the cylinder head 15 is not hindered by the water temperature sensor 65.

The pipe-shaped detection portion 64a of the exhaust characteristics sensor 64 is inserted downward into the collecting portion 61b from the upper surface of the exhaust manifold 61. Therefore, even if an exhaust gas containing moisture enters the inside of the detection portion 64a from the openings 64b, the moisture is prevented from remaining in the detection portion 64a to minimize the influence of the moisture on the detection accuracy. Further, the exhaust manifold 61 protrudes to the head cover 16 so that the exhaust manifold 61 is going away from the cylinder block 11, and the exhaust characteristics sensor 64 is provided on the upper surface of the exhaust manifold 61 which is remote from the cylinder block 11. Therefore, it is possible to define a space for receiving auxiliary machines, such as an oil filter 66 and a generator 67, on the side surface of the cylinder block 11, while securing a capacity of the exhaust manifold 61 to ensure a smooth flow of the exhaust gas. Furthermore, the water temperature sensor 65 is provided at the position lower than two joints 61d and 61e provided at the upper portion of the exhaust manifold cooling water jacket JH2. Therefore, it is possible to ensure that bubbles have difficulty staying in the upper portion of the exhaust manifold cooling water jacket JH2, thus enhancing the detection accuracy of the water temperature sensor 65.

Although the preferred embodiment of the present invention has been described above, it should be noted that various changes can be made without departing from the scope of the invention.

For example, the sensors, that is, the exhaust characteristics sensor 64 and the water temperature sensor 65, have been described, but the present invention is applicable to any other desired sensors, and the number of sensors may be three or more.

What is claimed is:

1. An outboard engine system comprising:
a cylinder head that forms a part of a combustion chamber; and
an exhaust passage component fixed to the cylinder head using a bolt,
wherein an exhaust passage, in communication with a combustion chamber, and a water jacket, having cooling water passing therethrough, are formed inside the exhaust passage component, and
wherein a plurality of sensors are mounted to an upper surface of the exhaust passage component and at least one side surface of the exhaust passage component, the side surface being different from a mounting surface to the cylinder head.

2. The outboard engine system according to claim 1, wherein the sensor mounted to the upper surface of the exhaust passage component is an exhaust characteristics sensor which detects characteristics of an exhaust gas flowing through the exhaust passage, and wherein the exhaust characteristics sensor includes a pipe-shaped detection portion inserted downward into the exhaust passage and which has an opening in the side surface.

3. The outboard engine system according to claim 2, wherein the exhaust passage component protrudes in a direction away from the cylinder block, and wherein the exhaust characteristics sensor is mounted to the upper surface of the exhaust passage component at a position remote from the cylinder block.

4. The outboard engine system according to claim 1, wherein the sensor mounted to the side surface of the exhaust passage component is a water temperature sensor for detecting temperature of the cooling water flowing through the water jacket.

5. The outboard engine system according to claim 4, wherein a cooling water outlet port is provided at an upper portion of the water jacket of the exhaust passage component, and wherein the water temperature sensor is mounted below the cooling water outlet port.
